(19) United States
(12) Patent Application Publication
(10) Pub. No.: US 2008/0103195 A1
(43) Pub. Date: May 1, 2008

(54) COMPOUNDS FROM ANTRODIA CAMPHORATA FOR INHIBITING THE GROWTH OF CANCER TUMOR CELLS

(76) Inventors: Sheng-Yun Liu, Danshuei Township (TW); Wu-Che Wen, Danshuei Township (TW); Wan-Ling Tsou, Danshuei Township (TW); Mao-Tien Kuo, Danshuei Township (TW)

Correspondence Address: BIRCH STEWART KOLASCH & BIRCH, PO BOX 747, FALLS CHURCH, VA, 22040-0747, US

(21) Appl. No.: 11/749,659

(22) Filed: May 16, 2007

(30) Foreign Application Priority Data
Oct. 31, 2006 (TW) 095140243

(51) Int. Cl. A61K 31/36; A61P 35/00

(52) U.S. Cl. 514/464

(57) ABSTRACT

The present invention relates a compound for inhibiting the growth of tumor cells. More specifically, the compound of the present invention is 4,7-dimethoxy-5-methy-1,3-benzodioxole. 4,7-dimethoxy-5-methy-1,3-benzodioxole of the present invention is used for inhibiting the growth of tumor cells of breast cancer, liver cancer, and prostate cancer, and the pharmaceutical composition comprises an effective dose of 4,7-dimethoxy-5-methy-1,3-benzodioxole.

### BACKGROUND OF THE INVENTION

1. Field of the Invention

The present invention relates a method of using a compound for inhibiting the growth of tumor cells. More specifically, the compound of the present invention is isolated from Antrodia camphorata extract.

2. The Prior Arts

Antrodia camphorata (Niu-chang-chih) known as “chang-ehih”, “niu-chang-ku”, “red chang”, “red chang-chih” or “chang-ku”, is a species only from Taiwan wherein “chih” means Ganoderma-like fungus and “ku” in Chinese means mushroom. Antrodia camphorata only grows on the inner heartwood wall of the endemic evergreen Cinnamomun kanehirai (Hay) (Lauraceae) from an altitude of 450 m to 2000 in Taiwan. Therefore, the fruiting bodies of Antrodia camphorata grow from the inner wall of the Cinnamomun kanehirai (Hay). The Cinnamomun kanehirai (Hay) species is restricted to mountain mange of Taoyuan Country and Nantou County. Since this endemic tree species is illegally logged, nowadays this endemic tree species is becoming rare and has been protected by the government so the wild Antrodia camphorata living within the endemic tree is extremely rare and difficult to obtain. And the growth of Antrodia camphorata is extremely slow and the growth period thereof is only from June to October, therefore Antrodia camphorata is very expensive in Taiwan.

The fruiting body of Antrodia camphorata is perennial, sessile, suberin or woody and has various shapes such as plate-shaped, bell-shaped, horseshoe-shaped, or tower-shaped. The fruiting body appears in flake-shaped on the wood surface, and when the fruiting body gets old, the front edges of the fruiting body will arise to roll into plate-shaped (lamellar) or dripstone-shaped. The top surface of Antrodia camphorata is from brown to dark color, and the wrinkle thereof is not clear. Antrodia camphorata is lustrous and has a flat and blunt edge, and the porous side of Antrodia camphorata is red-orange or partial yellow color.

Antrodia camphorata has a strong sassafras flavor and a strong bitter taste, and dried Antrodia camphorata is brownish white color. Antrodia camphorata in Taiwan is used for detoxification, liver protection, and anticancer. Antrodia camphorata as folk medicine has various bioactive compositions including polysaccharides such as β-glucan; triterpenoids, superoxide dismutase (SOD), adenosine, protein such as immunoglobulin; vitamin such as vitamin B and nicotinic acid; micromineral such as calcium, phosphate, and germanium; nucleic acids, lectins, steroids, lignins, and blood pressure stabilizers such as antodia acid. These bioactive compositions are believed to have beneficial effects such as antitumor, immunity enhancement, anti-allergic function, inhibition of platelet aggregation, antivirus activity, anti-bacteria activity, antihypertensive effect, blood-glucose lowering effect cholesterol-lowering effect, and liver protection.

Recently, Cherng et al. indicated a finding of three new triterpenoids named antcin A, antcin B, and antcin C in the fruiting body of Antrodia camphorata. These three new triterpenoids are compounds having ergostane skeletons (Cherng, I. H., and Chiang, H. C. 1995. Three new triterpenoids from Antrodia cinnamomea. J. Nat. Prod. 58:365-371). Chen et al. found the extract of the fruiting body obtained by using of ethyl alcohol has three triterpenoids named zhankuic acid A, zhankuic acid B, and zhankuic acid C (Chen, C. H., and Yang, S. W. 1995. New steroid acids from Antrodia cinnamomea,—a fungus parasitic on Cinnamomum micranthum. J. Nat. Prod. 58:1655-1661). Furthermore, Chiang et al. reported a finding of three new triterpenoids named antrocin, 4,7-dimethoxy-5-methy-1,3-benzodioxole, and 2,2′, 5,5′-teramethoxy-3,4,3′,4′-bi-methylenedioxy-6,6′-dimethylbiphenyl derived from sesquiterpene lactones and bisphenol in the fruiting body of Antrodia camphorata (Chiang, H. C., Wu, D. P., Cherng, I. W., and Ueng, C. H. 1995. A sesquiterpene lactone, phenyl and biphenyl compounds from Antrodia cinnamomea. Phytochemistry. 39:613-616). In 1996, Cherng et al. used the same method to obtain four new triterpenoids named antcin E, antcin F, methyl antcinate G, and methyl antcinate H (Cherng, I. H., Wu, D. P., and Chiang, H. C. 1996. Triteroenoids from Antrodia cinnamomea. Phytochemistry. 41:263-267). Yang et al. also found two new compounds having ergostane skeletons named zhankuic acid D and zhankuic acid E, and three compounds having lanostane skeletons named 15 α-acetyl-dehydrosulphurenic acid, dehydroeburicoic acid, and dehydrasulphurenic acid (Yang, S. W., Shen, Y. C., and Chen, C. H. 1996. Steroids and triterpenoids of Antrodia cinnamomea—a fungus parasitic on Cinnamomum micranthum. Phytochemistry. 41:1389-1392). Although the above experiments indicated Antrodia camphorata extract could inhibit cancers, the active compositions functioning to the tumor cells, though not identified. Accordingly, it would be desirable if such an active composition purified from Antrodia camphorata extract has a certain effect on human cancers.

### SUMMARY OF THE INVENTION

To understand which composition in Antrodia camphorata extract has the inhibitory effect on cancers, the Present invention relates a compound having the below structural formula (I), which is isolated from Antrodia camphorata extract,

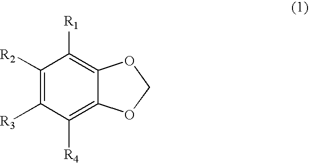

wherein R₁, R₂, R₃, and R₄ are each independently selected from methoxy, methyl, and hydrogen.

The molecular formula of the structural formula (I) is C₁₀O₄H₁₂. a molecular weight thereof is 196 Dalton, and the structural formula (I) occurs as slightly yellow granules and further comprises the structural formula (2), (3), (4), (5) (6), and (7) as below:

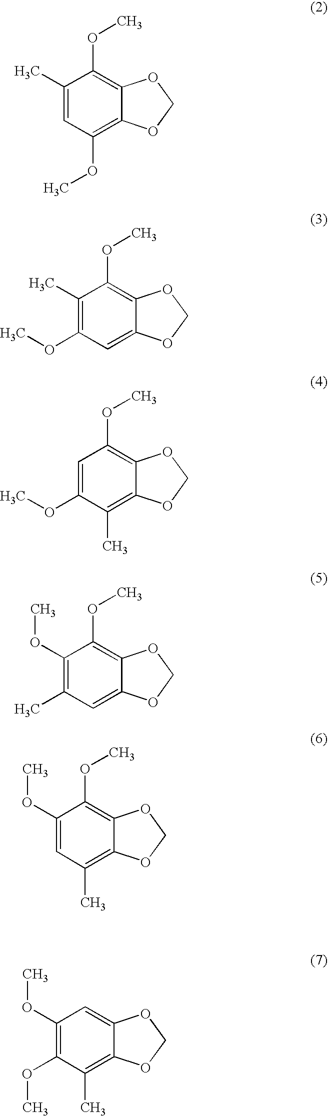

The structural formula (2) is 4,7-dimethoxy-5-methy-1,3-benzodioxole, the structural formula (3) is 4,6-dimethoxy-5-methy-1,3-benzodioxole, the structural formula (4) is 4,6-dimethoxy-7-methy-1,3-benzodioxole, the structural formula (5) is 4,5-dimethoxy-6-methy-1,3-benzodioxole, the structural formula (6) is 4,5-dimethoxy-7-methy-1,3-benzodioxole, and the structural formula (7) is 5,6-dimethoxy-4-methy-1,3-benzodioxole.

The present invention provides a method of using the above compounds for inhibiting the growth of tumor cells. More specifically, the above compounds can be contained in a pharmaceutical composition. The method of using the compound of the present invention for inhibiting the growth of tumor cells of breast cancer, liver cancer, and prostate cancer is by suppressing rapid growth of tumor cells and inhibiting tumor cell proliferation. Then, the method of using the compound of the present invention can slow progression of tumor. One preferred compound of the above compounds is 4,7-dimethoxy-5-methy-1,3-benzodioxole (i.e. the structural formula (2)).

The present invention provides a method of using a compound having the structural formula (I). The method comprises administrating an effective dose of the compound of the present invention.

The present invention further provides a method of using a pharmaceutical composition having the structural formula (1) for treating breast cancer, liver cancer, and prostate cancer, and the pharmaceutical composition comprises an effective dose of the compound.

The compound having the structural formula (1) of the present invention is isolated from an aqueous extract or an organic solvent extract of Antrodia camphorata. The organic solvent may include, but is not limited to, alcohols (e.g., methyl alcohol, ethyl alcohol, and propyl alcohol), esters (e.g., acetic acid ethyl ester), alkanes (e.g., hexane), and alkyl halides (e.g., chloromethane and chloroethane). Preferably, the organic solvent is from alcohols.

Although the present invention has been described below with reference to the preferred embodiment thereof, it is apparent to those skilled in the art that a variety of modifications and changes may be made without departing from the scope of the present invention which is intended to be defined by the appended claims.

### DETAILED DESCRIPTION OF THE PREFERRED EMBODIMENT

First, an aqueous extract or an organic solvent extract of Antrodia camphorata can be extracted from the mycelium, the fruiting body or the mixture of mycelium and fruiting body from Antrodia camphorata by using the methods of aqueous-organic solvent extraction as is well known in the art. The organic solvent may include, but is not limited to, alcohols (e.g., methyl alcohol, ethyl alcohol, and propyl alcohol), esters (e.g., acetic acid ethyl ester), alkanes (e.g., hexane), and alkyl halides (e.g., chloromethane and chloroethane). Preferably, the organic solvent is from alcohols. More preferably, the organic solvent is ethyl alcohol.

The aqueous extract or the organic solvent extract of Antrodia camphorata are further isolated and purified by using high-performance liquid chromatography to obtain the fractions. Each fraction is tested the inhibitory effect of cancer cell growth, and effective compositions in these fractions that can inhibit cancer cell growth are analyzed. Subsequently, the effective compositions are tested to inhibit the growth of different cancer tumor cells, and finally, the effect of the compound as the structural formula (I) to inhibit the growth of different cancer tumor cells is detected.

The present invention will be apparent to those skilled in the art by using 4,7-dimethoxy-5-methy-1, and 3-benzodioxole as the compound of the present invention. To confirm that 4,7-dimethoxy-5-methy-1, and 3-benzodioxole has the inhibitory effect on tumor cells such as breast cancer, liver cancer, and prostate cancer, the MTT assay, anti-tumor agents screening model of National Cancer Institute (NCI) of the United States National Institutes of Health, is used to measure the percentage of viable cells. It is indicated that 4,7-dimethoxy-5-methy-1, and 3-benzodioxole can reduce the percentage of viable cells of breast cancer tumor cells (MCF-7 and MDA-MB-231), liver cancer tumor cells (Hep 3B and Hep G2), and prostate cancer tumor cells (LNCaP and DU-145), and also reduce the 50% of the cell death (i.e. IC₅₀ value) of cancers. Therefore, 4,7-dimethoxy-5-methy-1,3-benzodioxole is capable of inhibiting cell proliferation of tumor cells such as breast cancer, liver cancer, and prostate cancer. The detailed description of preferred embodiments of the present invention is as follows.

### Embodiment 1

In Vitro Assay of Anti-Tumor Activity of Breast Cancer

According to anti-tumor agents screening model of National Cancer Institute (NCI) of the United States National Institutes of Health, the assay is processed by adding 4,7-dimethoxy-5-methy-1,3-benzodioxole into the culture medium of MCF-7 human tumor cells and MDA-MB-231 human tumor cells respectively. The assay of tumor cell viability can be evaluated by using the conventional MTT assay, and MCF-7 and MDA-MB-231 are human breast cancer tumor cell lines.

MTT assay is a conventional assay used to analyze the cell proliferation, percentage of viable cells, and cytotoxicity. MTT (3-(4,5-dimethylthiazole-2-yl)-2,5-diphenyl tetrazolium bromide) is a yellow dye, and is metabolized only in living cells by the mitochondrial succinate-tetrazolium reductase system to produce blue violet insoluble formazan products, thereby providing a measure of the number of viable cells upon generation of formazan products in viable cells.

First, MCF-7 human breast cancer cells and MDA-MB-231 human breast cancer cells were respectively cultured in culture medium supplemented with fetal bovine serum for 24 hours. The proliferative cells were washed with PBS once, and treated with one-fold trypsin/EDTA solution. After centrifuged at 1,200 rpm for 5 min, the supernatant was removed and the cell pellet was transferred to new tubes and treated with 10 ml medium to suspend the cells again, and then the cells were plated in 96 well microplates. Doses of ethyl alcohol extract of Antrodia camphorata (the control group) were respectively 30, 10, 3, 1, 0.3, 0.1, and 0.03 μg/ml for each well and 4,7-dimethoxy-5-methy-1,3-benzodioxole (the experimental group) were respectively 30, 10, 3, 1, 0.3, 0.1, and 0.03 μg/ml for each well incubated at 37° C., 5% CO₂ for 48 hours. 2.5 mg/ml MTT dye is added to each well without light and incubated for 4 hours, then treated with 100 μl lysis buffer to stop the reaction. Subsequently, absorption was measured on an enzyme immunoassay analyzer at 570 nm for the measurement of viable cell number. The concentration of the drug measured at 50% of the cell death (i.e. IC₅₀ value) was determined and calculated. The IC₅₀ values of the control group and the experimental group are summarized in Table 1.

TABLE 1
The result of in vitro assay of anti-tumor activity of breast cancer

| Sample | IC₅₀ (μg/ml) |
|---|---|
| Control group (Antrodia camphorata | |
| extract) | |
| MCF-7 | 11.461 |
| MDA-MB-231 | 26.812 |
| Experimental group (4,7-dimethoxy-5- | |
| methy-1,3-benzodioxole) | |
| MCF-7 | 1.721 |
| MDA-MB-231 | 0.992 |

Table 1 shows 4,7-dimethoxy-5-methy-1,3-benzodioxole inhibited MCF-7 human breast cancer cells and MDA-MB-231 human breast cancer cells with IC₅₀ of 1.721 μg/ml and 0.992 μg/ml respectively. The above IC₅₀ values are less than the IC₅₀ value of Antrodia camphorata extract. Therefore, it is confirmed that 4,7-dimethoxy-5-methy-1,3-benzodioxole from Antrodia camphorata extract is capable of inhibiting cell proliferation of breast cancer.

### Embodiment 2

In vitro activity assay of adjuvant treatment of breast cancer tumor cells

The activity assay was assessed according to in vitro anti-tumor agents screening model of National Cancer Institute (MCI). First, MCF-7 human breast cancer cells and MDA-MB-231 human breast cancer cells were respectively cultured in culture medium supplemented with fetal bovine serum for 24 hours. The proliferative cells were washed with PBS once, and treated with one-fold trypsin/EDTA solution. After centrifuged at 1,200 rpm for 5 min, the supernatant was removed and the cell pellet was transferred to new tubes and treated with 10 ml medium to suspend the cells again. Before the assay, the cells were treated with 0.0017 g/ml Taxol for 72 hours, and then plated in 96 well microplates. Doses of 4,7-dimethoxy-5-methy-1,3-benzodioxole were 30, 10, 3, 1, 0.3, 0.1, and 0.03 μg/ml (the experimental group) and 0 μg/ml of 4,7-dimethoxy-5-methy-1,3-benzodioxole (the control group) for each well incubated at 37° C., 5% CO₂ for 48 hours. 2.5 mg/ml MTT dye is added to each well without light and incubated for 4 hours, then treated with 100 μl lysis buffer to stop the reaction. Subsequently, absorption was measured on an enzyme immunoassay analyzer at 570 nm for the measurement of viable cell number. The concentration of the drug measured at 50% of the cell death (i.e. IC₅₀ value) was determined and calculated. The IC₅₀ values of the control group and the experimental group are summarized in Table 2.

TABLE 2
The result of in vitro assay of adjuvant treatment using Taxol
for breast cancer tumor cells

| Sample | Result |
|---|---|
| Control group | The ratio of viable cells (%) |
| MCF-7 (0.0017 μg/ml Taxol) | 69 ± 1 |
| MDA-MB-231 (0.0017 μg/ml Taxol) | 86 ± 1 |
| Experimental group | IC₅₀ (μg/ml) |
| MCF-7 (0.0017 μg/ml Taxol and 4,7- | 0.0007 |
| dimethoxy-5-methy-1,3- | |
| benzodioxole) | |
| MDA-MB-231 (0.0017 μg/ml Taxol | 0.0009 |
| and 4,7-dimethoxy-5-methy-1,3- | |
| benzodioxole) | |

Table 2 shows synergistic effects of Taxol on 4,7-dimethoxy-5-methy-1,3-benzodioxole inhibited MCF-7 human breast cancer cells and MDA-MB-231 human breast cancer cells results in low IC50 of 0.0007 μg/ml and 0.0009 μg/ml respectively. Therefore, it is confirmed that 4,7-dimethoxy-5-methy-1,3-benzodioxole from Antrodia camphorate extract is capable of inhibiting cell proliferation of breast cancer, and has more inhibitory effects with synergistic effects of Taxol.

### Embodiment 3

In Vitro Assay of Anti-Tumor Activity of Liver Cancer

According to anti-tumor agents screening model of National Cancer Institute (NCI) of the United States National Institutes of Health, the assay is processed by adding 4,7-dimethoxy-5-methy-1,3-benzodioxole into the culture medium of Hep 3B and Hep G2 human liver cancer tumor cells respectively.

First Hep 3B human liver cancer cells and Hep G2 human liver cancer cells were respectively cultured in culture medium supplemented with fetal bovine serum for 24 hours. The proliferative cells were washed with PBS once, and treated with one-fold trypsin/EDTA solution. After centrifuged at 1,200 rpm for 5 min, the supernatant was removed and the cell pellet was transferred to new tubes and treated with 10 ml medium to suspend the cells again, and then the cells were plated in 96 well microplates. Doses of ethyl alcohol extract of Antrodia camphorate (the control group) were respectively 30, 10, 3, 1, 0.3, 0.1, and 0.03 μg/ml for each well and 4,7-dimethoxy-5-methy-1,3-benzodioxole (the experimental group) were respectively 30, 10, 3, 1, 0.3, 0.1, and 0.03 μg/ml for each well incubated at 37° C., 5% CO₂ for 48 hours. 2.5 mg/ml MTT dye is added to each well without light and incubated for 4 hours, then treated with 100 μl lysis buffer to stop the reaction. Subsequently, absorption was measured on an enzyme immunoassay analyzer at 570 nm for the measurement of viable cell number. The concentration of the drug measured at 50% of the cell death (i.e. IC₅₀ value) was determined and calculated. The IC₅₀ values of the control group and the experimental group are summarized in Table 3.

TABLE 3
The result of in vitro assay of anti-tumor activity of liver cancer

| Sample | IC₅₀ (μg/ml) |
|---|---|
| Control group (Antrodia camphorata | |
| extract) | |
| Hep 3B | 6.112 |
| Hep G2 | 18.931 |
| Experimental group (4,7-dimethoxy-5- | |
| methy-1,3-benzodioxole) | |
| Hep 3B | 0.016 |
| Hep G2 | 2.462 |

Table 3 shows 4,7-dimethoxy-5-methy-1,3-benzodioxole inhibited Hep 3B human liver cancer cells and Hep O₂ human liver cancer cells with IC₅₀ of 0.016 μg/ml and 2.462 μg/ml respectively. The above IC₅₀ values are less than the IC₅₀ value of Antrodia camphorata extract. Therefore, it is confirmed that 4,7-dimethoxy-5-methy-1,3-benzodioxole from Antrodia camphorata extract is capable of inhibiting cell proliferation of liver cancer.

### Embodiment 4

In Vitro Activity Assay of Adjuvant Treatment of Liver Cancer Tumor Cells

The activity assay was assessed according to in vitro anti-tumor agents screening model of National Cancer Institute (MCI). First, Hep 3B human liver cancer cells and Hep G2 human liver cancer cells were respectively cultured in culture medium supplemented with fetal bovine serum for 24 hours. The proliferative cells were washed with PBS once, and treated with one-fold trypsin/EDTA solution. After centrifuged at 1,200 rpm for 5 min, the supernatant was removed and the cell pellet was transferred to new tubes and treated with 10 ml medium to suspend the cells again. Before the assay, the Hep 3B cell line was treated with 0.0043 μg/ml Lovastatin for 72 hours and the Hep G2 cell line was treated with 0.0017 μg/ml Taxol for 72 hours, and then plated in 96 well microplates. Doses of 4,7-dimethoxy-5-methy-1,3-benzodioxole were 30, 10, 3, 1, 0.3, 0.1, and 0.03 μg/ml (the experimental group) and 0 μg/ml of 4,7-dimethoxy-5-methy-1,3-benzodioxole (the control group) for each well incubated at 37° C., 5% CO₂ for 48 hours. 2.5 mg/ml MTT dye is added to each well without light and incubated for 4 hours, then treated with 100 μl lysis buffer to stop the reaction. Subsequently, absorption was measured on an enzyme immunoassay analyzer at 570 nm for the measurement of viable cell number. The concentration of the drug measured at 50% of the cell death (i.e. IC₅₀ value) was determined and calculated. The IC₅₀ values of the control group and the experimental group are summarized in Table 4.

TABLE 4
The result of in vitro assay of adjuvant treatment using
Lovastatin and Taxol for liver cancer tumor cells

| Sample | Result |
|---|---|
| Control group | The ratio of viable cells (%) |
| Hep 3B (0.0043 μg/ml Lovastatin) | 69 ± 1 |
| Hep G2 (0.0017 μg/ml Taxol) | 86 ± 1 |
| Experimental group | IC₅₀ (μg/ml) |
| Hep 3B (0.0043 μg/ml Lovastatin and 4,7- | 0.0007 |
| dimethoxy-5-methy-1,3-benzodioxole) | |
| Hep G2 (0.0017 μg/ml Taxol and 4,7- | 0.0129 |
| dimethoxy-5-methy-1,3-benzodioxole) | |

Table 4 shows synergistic effects of Lovastatin and Taxol on 4,7-dimethoxy-5-methy-1,3-benzodioxole inhibited Hep 3B human liver cancer cells and Hep G2 human liver cancer cells results in low IC₅₀ of 0.0007 μg/ml and 0.0129 μg/ml respectively. Therefore, it is confirmed that 4,7-dimethoxy-5-methy-1,3-benzodioxole from Antrodia camphorata extract is capable of inhibiting cell proliferation of liver cancer, and has more inhibitory effects with synergistic effects of Lovastatin and Taxol.

### Embodiment 5

In Vitro Assay of Anti-Tumor Activity of Prostate Cancer

According to anti-tumor agents screening model of National Cancer Institute (NCI) of the United States National Institutes of Health, the assay is processed by adding 4,7-dimethoxy-5-methy-1,3-benzodioxole into the culture medium of LNCaP and DU-145 human prostate cancer tumor cells respectively.

First, LNCaP human prostate cancer cells and DU-145 human prostate cancer cells were respectively cultured in culture medium supplemented with fetal bovine serum for 24 hours. The proliferative cells were washed with PBS once, and treated with one-fold trypsin/EDTA solution. After centrifuged at 1,200 rpm for 5 min, the supernatant was removed and the cell pellet was transferred to new tubes and treated with 10 ml medium to suspend the cells again, and then the cells were plated in 96 well microplates. Doses of ethyl alcohol extract of Antrodia camphorata (the control group) were respectively 30, 10, 3, 1, and 0.3 μg/ml for each well and 4,7-dimethoxy-5-methy-1,3-benzodioxole (the experimental group) were respectively 30, 10, 3, 1, and 0.3 μg/ml for each well incubated at 37° C., 5% CO₂ for 48 hours. 2.5 mg/ml MTT dye is added to each well without light and incubated for 4 hours, then treated with 100 μl lysis buffer to stop the reaction. Subsequently, absorption was measured on an enzyme immunoassay analyzer at 570 nm for the measurement of viable cell number. The concentration of the drug measured at 50% of the cell death (i.e. IC₅₀ value) was determined and calculated. The IC₅₀ values of the control group and the experimental group are summarized in Table 5.

TABLE 5
The result of in vitro assay of anti-tumor activity of prostate cancer

| Sample | IC₅₀ (μg/ml) |
|---|---|
| Control group (Antrodia camphorata | |
| extract) | |
| LNCaP | 45.47 |
| DU-145 | 30.15 |
| Experimental group (4,7-dimethoxy-5- | |
| methy-1,3-benzodioxole) | |
| LNCaP | 4.46 |
| DU-145 | 2.21 |

Table 5 shows 4,7-dimethoxy-5-methy-1,3-benzodioxole inhibited LNCaP human prostate cancer cells and DU-145 human prostate cancer cells with IC₅₀ of 4.46 μg/ml and 2.21 μg/ml respectively. The above IC₅₀ values are less than the IC₅₀ value of Antrodia camphorata extract. Therefore, it is confirmed that 4,7-dimethoxy-5-methy-1,3-benzodioxole from Antrodia camphorata extract is capable of inhibiting cell proliferation of prostate cancer.

### Embodiment 6

In Vitro Activity Assay of Adjuvant Treatment of Prostate Cancer Tumor Cells

The activity assay was assessed according to in vitro anti-tumor agents screening model of National Cancer Institute (MCI). First, LNCaP human prostate cancer cells and DU-145 human prostate cancer cells were respectively cultured in culture medium supplemented with fetal bovine serum for 24 hours. The proliferative cells were washed with PBS once, and treated with one-fold trypsin/EDTA solution. After centrifuged at 1,200 rpm for 5 min, the supernatant was removed and the cell pellet was transferred to new tubes and treated with 10 ml medium to suspend the cells again. Before the assay, the LNCaP cell line was treated with 0.0017 μg/ml Taxol for 72 hours and the DU-145 cell line was treated with 0.0043 μg/ml Taxol for 72 hours, and then plated in 96 well microplates. Doses of 4,7-dimethoxy-5-methy-1,3-benzodioxole were 30, 10, 3, 1, 0.3, 0.1, and 0.03 μg/ml (the experimental group) and 0 μg/ml of 4,7-dimethoxy-5-methy-1,3-benzodioxole (the control group) for each well incubated at 37° C., 5% CO₂ for 48 hours. 2.5 mg/ml MTT dye is added to each well without light and incubated for 4 hours, then treated with 100 μl lysis buffer to stop the reaction. Subsequently, absorption was measured on an enzyme immunoassay analyzer at 570 nm for the measurement of viable cell number. The concentration of the drug measured at 50% of the cell death (i.e. IC₅₀ value) was determined and calculated. The IC₅₀ values of the control group and the experimental group are summarized in Table 6.

TABLE 6
The result of in vitro assay of adjuvant treatment using
Taxol for prostate cancer tumor cells

| Sample | Result |
|---|---|
| Control group | The ratio of viable cells (%) |
| LNCaP (0.0017 μg/ml Taxol) | 55 ± 1 |
| DU-145 (0.0043 μg/ml Taxol) | 71 ± 1 |
| Experimental group | IC₅₀ (μg/ml) |
| LNCaP (0.0017 μg/ml Lovastatin and 4,7- | 1.16 |
| dimethoxy-5-methy-1,3-benzodioxole) | |
| DU-145 (0.0043 μg/ml Taxol and 4,7- | 0.71 |
| dimethoxy-5-methy-1,3-benzodioxole) | |

Table 6 shows synergistic effects of Taxol on 4,7-dimethoxy-5-methy-1,3-benzodioxole inhibited LNCaP human prostate cancer cells and DU-145 human prostate cancer cells results in low IC₅₀ of 1.16 μg/ml and 0.71 μg/ml respectively. The above IC₅₀ values are less than the IC₅₀ value of Antrodia camphorata extract. Therefore, it is confirmed that 4,7-dimethoxy-5-methy-1,3-benzodioxole from Antrodia camphorata extract is capable of inhibiting cell proliferation of prostate cancer, and has more inhibitory effects with synergistic effects of Taxol.

What is claimed is:

1. A method of using a compound having the below structural formula, comprising: administering an effective dose for inhibiting the growth of breast cancer tumor cells, 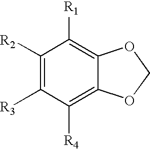
wherein R₁, R₂, R₃, and R₄ are each independently selected from methoxy, methyl, and hydrogen.

2. The method as claimed in claim 1, wherein the compound is isolated from Antrodia camphorata extract.

3. The method as claimed in claim 2, wherein the compound is isolated from an aqueous extract of Antrodia camphorate.

4. The method as claimed in claim 2, wherein the compound is isolated from an organic solvent extract of Antrodia camphorate.

5. The method as claimed in claim 4, wherein the organic solvent is selected from the group consisting of esters, alcohols, alkanes, and alkyl halides.

6. The method as claimed in claim 5, wherein the alcohol is ethyl alcohol.

7. The method as claimed in claim 5, wherein the compound is 4,7-dimethoxy-5-methy-1,3-benzodioxole.

8. The method as claimed in claim 1, wherein the breast tumor cells are from a MCF-7 cell line or an MDA-MB-231 cell line.

9. The method as claimed in claim 7, wherein the breast tumor cells are from a MCF-7 cell line or an MDA-MB-231 cell line.

10. A method of using a compound having the below structural formula, comprising: administering an effective dose for inhibiting the growth of liver cancer tumor cells, 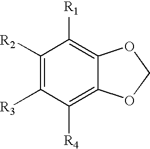
wherein R₁, R₂, R₃, and R₄ are each independently selected from methoxy, methyl, and hydrogen.

11. The method as claimed in claim 10, wherein the compound is isolated from Antrodia camphorate extract.

12. The method as claimed in claim 11, wherein the compound is isolated from an aqueous extract of Antrodia camphorata.

13. The method as claimed in claim 11, wherein the compound is isolated from an organic solvent extract of Antrodia camphorate.

14. The method as claimed in claim 13, wherein the organic solvent is selected from the group consisting of esters, alcohols, alkanes, and alkyl halides.

15. The method as claimed in claim 14, wherein the alcohol is ethyl alcohol.

16. The method as claimed in claim 10, wherein the compound is 4,7-dimethoxy-5-methy-1,3-benzodioxole.

17. The method as claimed in claim 10, wherein the liver tumor cells are from a Hep 3B cell line or an Hep G2 cell line.

18. The method as claimed in claim 16, wherein the liver tumor cells are from a Hep 3B cell line or an Hep G2 cell line.

19. A method of using a compound having the below structural formula, comprising: administering an effective dose for inhibiting the growth of prostate cancer tumor cells, 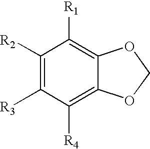
wherein R₁, R₂, R₃, and R₄ are each independently selected from methoxy, methyl, and hydrogen.

20. The method as claimed in claim 19, wherein the compound is isolated from Antrodia camphorata extract.

21. The method as claimed in claim 20, wherein the compound is isolated from an aqueous extract of Antrodia camphorate.

22. The method as claimed in claim 20, wherein the compound is isolated from an organic solvent extract of Antrodia camphorata.

23. The method as claimed in claim 22, wherein the organic solvent is selected from the group consisting of esters, alcohols, alkanes, and alkyl halides.

24. The method as claimed in claim 23, wherein the alcohol is ethyl alcohol.

25. The method as claimed in claim 19, wherein the compound is 4,7-dimethoxy-5-methy-1,3-benzodioxole.

26. The method as claimed in claim 19, wherein the prostate tumor cells are from an LNCaP cell line or a DU-145 cell line.

27. The method as claimed in claim 25, wherein the prostate tumor cells are from an LNCaP cell line or a DU-145 cell line.

28. A method of using a pharmaceutical composition having the below structural formula, comprising: administering an effective dose for inhibiting the growth of tumor cells, 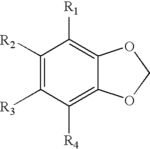
wherein R₁, R₂, R₃, and R₄ are each independently selected from methoxy, methyl, and hydrogen; and wherein the tumor cells are selected from the group consisting of breast cancer, liver cancer, and prostate cancer.
